(19) United States
(12) Patent Application Publication
(10) Pub. No.: US 2005/0260136 A1
(43) Pub. Date: Nov. 24, 2005

(54) Topical immune competency diagnostic compositions and methods of use

(76) Inventors: Leonard L. Kaplan, East Brunswick, NJ (US); William R. Levis, New York City, NY (US)

Correspondence Address: Leonard L. Kaplan, One Minute Man Court, East Brunswick, NJ, 08816, US

(21) Appl. No.: 10/849,703

(22) Filed: May 20, 2004

(51) Int. Cl. A61K 49/00

(52) U.S. Cl. 424/9.6; 424/9.81

(57) ABSTRACT

The compositions and methods of this invention are used to assess the level of immune competence of tested individuals based on the degree of immune response as a surrogate marker of CD4 T Cell lymphocytes as measured by the intensity of the resultant skin reaction score following controlled topical application of a unique anhydrous composition containing a contact sensitizer. The results of assessment are useful in determining appropriate treatment of immuno-compromised patients. Preferred compositions contain, but are not limited to, Diphenylcyclpropenone as a preferred embodiment of the class of contact sensitizing agents applied to the skin in an optimally prepared formulation preferably containing about 0.4% of the contact sensitizer.

### BACKGROUND OF THE INVENTION

The induction of skin reactions has been used for predictive testing in contact allergies. For example, Basketter and Kimber (full citation) describe allergic contact dermatitis resulting from skin sensitization wherein a contact allergen induces a cutaneous immune response that may elicit a cutaneous inflammatory reaction following subsequent exposure to the inducing allergen. Contact allergy testing, however, is limited to the estimation of relative allergenic potency potential of the tested chemical allergen.

It has been unexpectedly discovered that the skin reaction induced by the topical application of certain novel compositions containing a contact sensitizer in a carrier is indicative of the competency of the immune system in tested patients. These unique compositions are disclosed in U.S. Pat. No. 6,455,586 B 1 issued on Sep. 24, 2002, the entire contents of which are by reference fully incorporated herein.

As disclosed in the U.S. Pat. No. 6,455,586, the medical use of the preferred contact sensitizer diphenylcyclopropenone had been limited primarily to the treatment of alopecia areata as disclosed in referenced U.S. Pat. No. 4,985,464 wherein the volatile solvent acetone with unreliable absorption properties was used as the formulation vehicle.

The present invention provides an easy, inexpensive, non-invasive composition and method useful for the assessment of the level of immune competence of the individuals tested based on the degree of immune response as a surrogate marker of CD4 T Cell lymphocytes as measured by the intensity of the resultant skin reaction.

### SUMMARY OF THE INVENTION

This invention provides compositions for the topical application of contact sensitizers in a unique topical pharmaceutical emulsion drug delivery system to induce a skin reaction that is indicative of the competence of the patient's immune system. Topical immune diagnostic compositions serve as a non-invasive, safe, predictive test to assess cell mediated immune response function, particularly in immuno-compromised patients with diseases such as AIDS, Hepatitis, Smallpox, other viral diseases and certain forms of Cancer. Skin reaction scores are quantitated according to a skin reaction intensity scoring method and act as a surrogate marker of blood levels of CD4 T cell lymphocytes.

Topical compositions, containing contact sensitizing agents as the active components, can induce the development of a skin reaction based on stimulation of a cell-mediated immune response. The intensity of this skin reaction serves as a measure of immune competence in the tested individual that is based on correlation with blood levels of CD4 T cell lymphocytes.

The compositions contain controlled amounts of diphenylcyclopropenone, dinitrochlorobenzene, dinitrofluorobenzene, squaric acid dibutylester, urishiol, oxazolone, paraphenylenediamine or other medically useful contact sensitizers in a nontoxic delivery system formula consisting of pharmaceutically acceptable non-volatile, non-ionic surfactants and pharmaceutically acceptable emollients at optimized levels wherein the delivery system will not induce a skin reaction.

Results of a clinical study in 40 HIV sero-positive AIDS patients confirm the accuracy of these compositions with sensitivity scores of 86% and 100% specificity.

A preferred embodiment of the invention includes about 0.4% diphenylcyclopropenone as the contact sensitizer uniquely formulated in a micro emulsified delivery system consisting of the non-ionic surfactant polyoxyethylene 20 sorbitan monooleate and the emollients isopropyl myristate and/or isopropyl palmitate.

### DETAILED DESCRIPTION OF THE INVENTION

Diphenylcyclopropenone (DPC) in a topical delivery system applied topically to the forearm of HIV sero-positive AIDS patients can serve as a reliable surrogate marker of cell mediated immune competency based on: (A) its contact sensitizer immune skin reactivity results; and (B) the principle that individuals with blood counts of CD4 T-Cell counts above 300 cu/mm are considered immune competent and individuals with blood counts of CD4 T-Cells below 300 cu/mm are considered immune incompetent or immune compromised.

Clinical study findings confirmed that there are differences in the indicated activity of the tested dosage concentration of DPC; and that 0.4% DPC dosage concentration serves as the preferred topical effective concentration in distinguishing between >300 and <300 CD4 cu/mm blood counts as the break point between immune competency and immune incompetency in HIV sero-positive AIDS patients.

The compositions of this invention accordingly may contain non-ionic surfactants of the following classes:

Polyoxyethylene (POE) sorbitan fatty acid esters identified generically as POE 20 sorbitan monolaurate, POE 4 sorbitan monolaurate, POE 20 sorbitan monopalmitate, POE 20 sorbitan monostearate, POE 20 sorbitan monooleate, POE 5 sorbitan monooleate, POE 20 sorbitan trioleate and the like that are oily liquids with low vapor pressure properties and therefore non-volatile and non-irritating to the skin and have the property of emulsifying immiscible combinations of the active ingredient contact sensitizers and the emollient co-solvents embodied by the following alcoholic esters of myristic and palmitic fatty acids: Isopropyl myristate consisting of esters of isopropyl alcohol and saturated high molecular weight fatty acids, principally myriatic acid; and Isopropyl palmitate consisting of esters of isopropyl alcohol and saturated high molecular weight fatty acids, principally palmitic acid, and other like alcohol esters of saturated high molecular weight fatty acids that are mobile oily liquids at room temperature are miscibly emulsified with the polyoxyethylene sorbitan fatty acid esters embodied in this invention to provide nontoxic, non-volatile topical drug delivery vehicles for the contact sensitizers of this invention.

The drug compositions of this invention are comprised of the contact sensitizer contained in optimized pharmaceutical vehicles as described in the following illustrative examples:

| | | |
|---|---|---|
| (1) | Dinitrochlorobenzene | 0.001% |
| | Polyoxyethylene 20 sorbitan monolaureate | 50.000% |
| | Isopropyl palmitate | 49.999% |
| (2) | Diphenylcyclopropenone | 0.001% |
| | Polyoxyethylene 20 sorbitan monooleate | 50.000% |
| | Isopropyl myristate | 49.999% |
| (3) | Squaric Acid Dibutylester | 0.01% |
| | Polyoxyethylene 20 sorbitan.monopalmitate | 85.99% |
| | Isopropyl myristate | 14.00% |
| (4) | Squaric Acid Dibutylester | 1.00% |
| | Polyoxyethylene 4 sorbitan monolaurate | 99.00% |
| (5) | Diphenylcyclopropenone (DPC) | 0.05% |
| | Polyoxyethylene 20 sorbitan monooleate | 50.00% |
| | Isopropyl myristate | 49.05% |
| (6) | Diphenylcyclopropenone | 0.10% |
| | Polyoxyethylene 20 sorbitan monooleate | 50.00% |
| | Isopropyl myristate | 49.90% |
| (7) | Diphenylcyclopropenone | 0.20% |
| | Polyoxyethylene 20 sorbitan monooleate | 50.00% |
| | Isopropyl myristate | 49.80% |
| (8) | Diphenylcyclopropenone | 0.40% |
| | Polyoxyethylene 20 sorbitan monooleate | 50.00% |
| | Isopropyl myristate | 49.60% |

These composition examples are cited to demonstrate, but not to limit, various concentrations of active contact sensitizers in non-volatile vehicles applied to the skin. Other examples of effective contact sensitizers applicable to formulation in the unique non-volatile, non-irritating, skin absorbable vehicle compositions include, Oxazolone, Fluoroscine Isothiocyanate, Dinitrofluorobenzene, Beryllium, Nickel Chloride, Trinitrochlorobenzene, Urishiol, and Paraphenylenediamine. A preferred composition comprises the contact sensitizer Diphenylcyclopropenone in amounts greater than 0.05% weight to volume of said composition, preferably greater than or equal to about 0.2%, more preferably greater than or equal to 0.4% up to 1.0%.

For the purposes of this invention, the T cell CD4 count of 300 cu/mm is used as the boundary for distinguishing the immune competency of patients. Patients with CD4 counts less than 300 can be medically defined as immuno-incompetent or compromised; patients with CD4 counts higher than 300 can be medically defined as immuno-competent.

The following examples are provided for illustrative purposes only and are not to be interpreted as limiting the scope of this invention. The scope of this invention is defined by the attached claims.

### EXAMPLES

Subjects for the Examples:

For the examples, HIV sero-positive AIDS patients were tested based on HIV sero-positivity for a 28-day observational clinical trial. 40 subjects completed the study. Topical 0.1 ml applications of 0.4%, 0.2%, 0.1%, 0.05% Diphenylcyclopropenone in a proprietary anhydrous skin penetrating vehicle plus the vehicle placebo were made to the inner aspect of the forearms of patients and covered with a 1 inch adhesive bandage. Skin reactivity scores of a 4-point scaled based on induration and erythema were recorded weekly, as were side effects to the topical treatments.

Skin Reaction Scoring

For the examples, skin reaction scores were assigned using the following scale:

| Score | Indicia |
|---|---|
| 4+ | Blisters, unequivocal Spontaneous Flare at sensitizing |
| | and challenge dose sites |
| 3+ | Vesicles, Spontaneous Flare at sensitizing dose site only |
| 2+ | Cutaneous Induration Erythema |
| 1+ | Erythema only |
| 0 | No indicia |

For the examples, patients having a skin reaction score of +2, +3 and +4 were considered to have a positive response and are labeled as “Reactors”; patients having a skin reaction score of 0 and +1 were considered to not have a response and are labeled as “Non-Reactors”.

Skin Reactions Scores as indicators of immune function responses following single applications of 0.4%, 0.2%, 0.1% and 0.05% DPC compositions were used to distinguish between patients with T Cell CD4 blood counts less than 300 per cu/mm and patients with T Cell CD4 counts higher than 300 per cu/mm.

### Example 1

### Skin Reaction Scores in Patients with CD4 Blood Counts <300 cu/mm Right Forearm Application

| % DPC | # Reactor | # Non-Reactor | Total Number |
|---|---|---|---|
| Applied | Patients | Patients | of Patients |
| 0.4% | 0 (0%) | 4 (100%) | 4 |
| 0.2% | 3 (60%) | 2 (40%) | 5 |
| 0.1% | 0 (0%) | 2 (100%) | 2 |
| 0.05% | 0 (0%) | 2 (100%) | 2 |

Example 1. presents measures of Sensitivity of the topical DPC Solutions skin reactions in the HIV sero-positive test population with CD4 counts <300 cu/mm. In this regard, three patients treated with 0.2% DPC who did show positive skin reactions all had CD4 levels close to the 300 count immune competency break point. Importantly, none of the patients treated with 0.4% DPC showed positive skin reactions, confirming 0.4% DPC value as a sensitive predictor of CD4 counts below 300 and thereby defined as immune incompetent and in need of therapy.

### Example 2

### Skin Reaction Scores in Patients with CD4 Blood Counts >300 cu/mm—Right Forearm Application

| % DPC | # Reactor | # Non-Reactor | Total Number |
|---|---|---|---|
| Applied | Patients | Patients | of Patients |
| 0.4% | 6 (85.7%) | 1 (14.3%) | 7 |
| 0.2% | 5 (71.4%) | 2 (28.6%) | 7 |
| 0.1% | 1 (20%) | 4 (80%) | 5 |
| 0.05% | 0 (0%) | 8 (100%) | 8 |

Example 2. presents measures of specificity of the topical DPC Solutions based on positive skin reactions in the HIV sero-positive test population with CD4 counts >300 cu/mm. In this regard, only one patient subjected to 0.4% DPC was a non-skin reactor and this patient had a CD4 count of 319 close to the break point of 300 between immune competency and incompetency. And of the two non-skin reactor patients subjected to 0.2% DPC Solution, one had a CD4 count of 316 again close to the 300 break point. The only other patient had an outlier CD4 count of 587, and is considered as a false negative.

### Example 3

### Skin Reaction Scores—Vehicle Placebo Control

Patients with CD4 counts <300−N=14

Patients with CD4 Counts <300−N=26

Results: All 40 HIV sero-positive AIDS patients in the study were Non-Reactors to the Vehicle Placebo Applications.

The following Tables 1 and 2 summarize the results of the tests of Examples 1 and 2:

TABLE 1
Combine the Two Higher Dosages (0.4% and 0.2%) Scores and the
Two Lower Dosages (0.1% and 0.05%) Scores
Skin Reactions Scores in Patients with CD4 Blood Counts <300 cu/mm

| DPC | Reactors | Non-Reactors | Number of Patients |
|---|---|---|---|
| 0.4% + 0.2% | 3 (33%) | 6 (67%) | 9 |
| 0.1% + 0.05% | 0 (0%) | 4 (100%) | 4 |

TABLE 2
Combine the Two Higher Dosages (0.4% and 0.2%) Scores and the
Two Lower Dosages (0.1% and 0.05%) Scores
Skin Reaction Scores in Patients with CD4 Blood Counts >300 cu/mm

| DPC | Reactors | Non-Reactors | Number of Patients |
|---|---|---|---|
| 0.4% + 0.2% | 11 (78.6%) | 3 (21.4%) | 14 |
| 00.1% + 0.05% | 1 (7.7%) | 12 (92.3%) | 13 |

### SUMMARY

The data of the above examples were statistically analyzed for significance of differences among DPC % dosages topically applied to the forearms of immuno-incompetent or compromised (<300 CD4) and immuno-competent (>300 CD4) HIV sero-positive AIDS patients. These statistic results strongly suggest that 0.4% DPC contact sensitizer concentration applied topically can serve as a reliable surrogate marker of immune competency based on the premise that individuals with >300 CD4 T Cell cu/mm count demonstrate a measurable immune response skin reaction response; These data also demonstrate that lower 0.1% and 0.05% concentrations do not serve as strong markers of immune competence; and that 0.2% DPC concentration may serve as a threshold contact sensitizer, but not as effectively as does 0.4%.

Subjects with CD4 counts greater than 300 cu/mm showed positive 2+, 3+and 4+skin reactions with erythema and induration. Subjects with CD4 counts less than 300 cu/mm were non-skin reactive. This was found in 36 of the 40 subjects. One subject with CD4 levels of 319 cu/mm failed to show a skin reaction and 3 subjects with CD4 counts slightly less than 300 showed positive skin reactivity. Statistical Sensitivity was 87.4% and Specificity was 100% with the 0.4% Topical Diphenylcyclopropenone Solution selected as the optimal dosage concentration. Side effects were minimal and were limited to adenopathies that were self-limited and disappeared upon completion of the study. None of the subjects withdrew because of side effects.

### TREATMENT RECOMMENDATION

In clinical practice, the clinician would make a medical decision as to the need for a course of medical therapy based upon the skin reaction score. The following treatment protocol to titer need for high anti-retroviral therapy (HAART) in AIDS patients, based upon diagnostic testing using the compositions of the present invention can be established as follows:

| | |
|---|---|
| Skin Reaction Score 4+ | High responders - withhold HAART |
| Skin Reaction Score 3+ to 2+ | Moderate responders - question as to |
| | need for HAART give immune therapy |
| Skin Reaction Score 0 to 1+ | Low responders - give HAART (high |
| | antiretroviral therapy) |

1. A topical diagnostic composition for the evaluation of the competence of a patient's immune system comprising a contact sensitizer capable of producing an immune response at the site of application which is indicative of the patient's immune competency.

2. The composition of claim 1, said composition comprising:
a) a contact sensitizer selected from the group consisting of dinitrochlorobenzene, dinitrofluorobenzene, diphenylcyclopropenone, oxazolone, paraphenylenediamine, squaric acid dibutylester, and urushiol and
b) a carrier comprised of a first co-solvent selected from the group consisting of polyoxyethylene 20 sorbitan monolaurate, polyoxyethylene 4 sorbitan monolaurate, polyoxyethylene 20 sorbitan monopalmitate, polyoxyethylene 20 sorbitan monostearate, polyoxyethylene 20 sorbitan monooleate, polyoxyethylene 5 sorbitan monooleate, polyoxyethylene 20 sorbitan trioloeate, and combinations thereof and a second cosolvent selected from the group consisting of isopropyl myristate, isopropyl palmitate and combinations thereof

3. The composition of claim 1 wherein said contact sensitizer is diphenylcyclopropenone.

4. The composition of claim 1 wherein said contact sensitizer compromises from about 0.05% to about 1.0% weight to volume of said composition.

5. The composition of claim 1 wherein said first co-solvent is polyoxyethylene 20 sorbitan monolaurate or polyoxyethylene 20 sorbitan monooleate.

6. The composition of claim 1 wherein said second co-solvent is isopropyl myristate or isopropyl palmitate.

7. A method of evaluating the immune system competency of a patient comprised of topically applying an anhydrous, non-toxic absorbable composition wherein said composition produces an immune response at the site of application such that the degree of said immune response is a surrogate marker of the level of CD4 T Cell lymphocytes in the patient.

8. The method of claim 7, wherein said composition comprises a contact sensitizer selected from the group consisting of dinitrochlorobenzene, dinitrofluorobenzene, diphenylcyclopropenone, oxazolone, paraphenylenediamine, squaric acid dibutylester, and urushiol and a carrier comprised of a first co-solvent selected from the group consisting of polyoxyethylene 20 sorbitan monolaurate, polyoxyethylene 4 sorbitan monolaurate, polyoxyethylene 20 sorbitan monopalmitate, polyoxyethylene 20 sorbitan monostearate, polyoxyethylene 20 sorbitan monooleate, polyoxyethylene 5 sorbitan monooleate, polyoxyethylene 20 sorbitan trioloeate, and combinations thereof and a second co-solvent selected from the group consisting of isopropyl myristate, isopropyl palmitate and combinations thereof

9. The method of claim 7 wherein said contact sensitizer is diphenylcyclopropenone.

10. The method of claim 7 wherein said contact sensitizer compromises from about 0.05% to about 1.0.% weight to volume of said composition.

11. The method of claim 7 wherein said first co-solvent is polyoxyethylene 20 sorbitan monolaurate or polyoxyethylene 20 sorbitan monooleate.

12. The method of claim 7 wherein said second co-solvent is isopropyl myristate or isopropyl palmitate.

13. The method of claim 7 wherein said composition is first applied to the skin of said patient and secondly covered by an absorbent pad under an occlusive or semi-occlusive backing for localized administration of said contact sensitizer for absorption through the skin.

14. A method for determining the need for high anti-retroviral therapy (HAART) in AIDS patients, comprising topically applying an anhydrous, non-toxic absorbable composition to the skin of an AIDS patient; evaluating the immune response induced by the composition at the site of application; determining the level of HAART treatment for the patient based upon the evaluation of said immune response.

15. The method of claim 14 wherein said topical composition comprises a contact sensitizer.

16. The method of claim 15 wherein said contact sensitizer is diphenylcyclopropenone.
